(19) United States
(12) Patent Application Publication
(10) Pub. No.: US 2025/0281137 A1
(43) Pub. Date: Sep. 11, 2025

(54) Dental App

(71) Applicant: Ian Guyton, Lancaster, CA (US)

(72) Inventor: Ian Guyton, Lancaster, CA (US)

(21) Appl. No.: 18/600,695

(22) Filed: Mar. 9, 2024

(51) Int. Cl. A61B 6/51; G16H 40/67

(52) U.S. Cl. CPC A61B 6/512; G16H 40/67

(57) ABSTRACT

With this innovation, a person may be able to keep their teeth all the way into their senior citizen years. Parents can use this technology for themselves and their children. The present invention, herein referred to as “Dental App” comprises a device and associated software application which allows users to take their own dental x-ray and get from the app's database 5 possible diagnosis that the dental problem could be (cavity, gum disease, tartar, etc), from the comfort and convenience of their own home. The user then could email the results to their dentist for a professional opinion and speedy surgery if recommended. Simply said, I want “Dental App” users to get urgent treatment for dental problems.

### CROSS-REFERENCE TO RELATED APPLICATIONS

N/A.

### STATEMENT REGARDING FEDERALLY SPONSORED RESEARCH OR DEVELOPMENT

N/A yet.

### THE NAMES OF THE PARTIES TO A JOINT RESEARCH AGREEMENT

N/A yet.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

N/A yet.

### STATEMENT REGARDING PRIOR DISCLOSURES BY THE INVENTOR OR A JOINT INVENTOR

Disclosed idea to thoughtstopaper.com on around Mar. 17, 2021, patent pending expired on around Oct. 15, 2022.

### BACKGROUND OF THE INVENTION

Dental X-rays, or ‘Dental Radiographs’, are commonly used in dentistry for a variety of clinical reasons, such as identify hidden tooth damage or decay, benign masses, cavities, etc. The present invention introduces a proprietary new device and software app which allows users to effectively utilize a dental X-ray from the convenience of their own home, or anywhere they can access a computer.

### Field of the Invention

The present invention relates generally to dentistry. More specifically, it relates to an apparatus and software application for dental X-rays.

### Description of Related Art including information disclosed under 37 CFR 1.97 and 1.98

N/A.

### BRIEF SUMMARY OF THE INVENTION

The “Dental App” and device solves a problem of mobility and convenience. Two advantages are users can go to their dental appointment with a more informed opinion of their oral health needs (preventive and diagnostic), and this information can be obtained from the comfort and convenience of the user's home.

### DETAILED DESCRIPTION OF THE INVENTION

The present invention, herein referred to as, “Dental App”, comprises a device and associated software application which allows users to take their own dental X-ray from the comfort and convenience of their own home. The Dental App device serves as mouthpiece which attaches to a personal computer the same way a printer would. The Dental App software application comprises an encyclopedia so that after the user takes their dental X-ray, the app provides an analysis of 5 different diagnosis of what the potential problem may be, such as cavity, tarter, etc. The user can then email the results to a professional dentist for further treatment and analysis. This personal computer app and device has expansion potential for other personal computer X-ray applications, such as eye exams and hearing tests with devices and user-friendly software applications from the comfort and convenience of a user's personal computer.

Although the invention has been explained in relation to its preferred embodiment, it is to be understood that many other possible modifications and variations can be made without departing from the spirit and scope of the invention.

### BRIEF DESCRIPTION OF THE SEVERAL VIEWS OF THE DRAWING(S)

FIG. 1a shows a person taking a selfie, but instead, a user with the Dental App software and apparatus can take a dental X-ray from the comfort and convenience of their own home.
Image designed by Freepik.com.

FIG. 1b shows a dental X-ray is uploaded to a personal computer and then displayed on the Dental App with analysis of potential illnesses.

Image designed by home-dental.com.

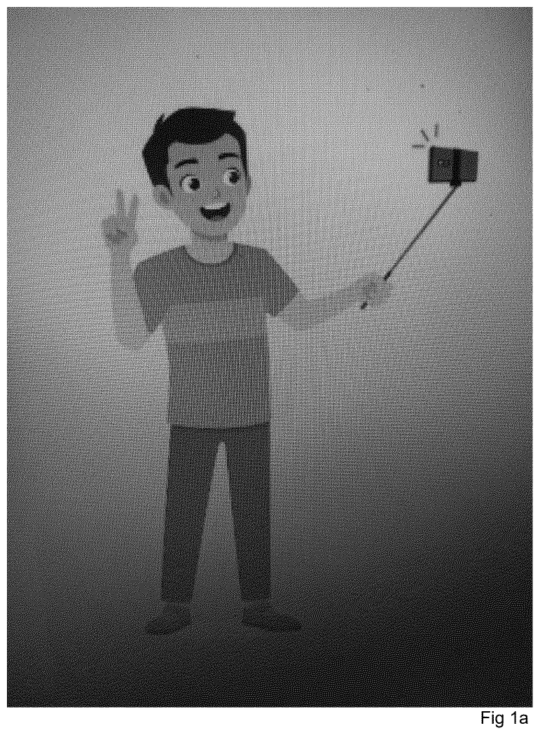

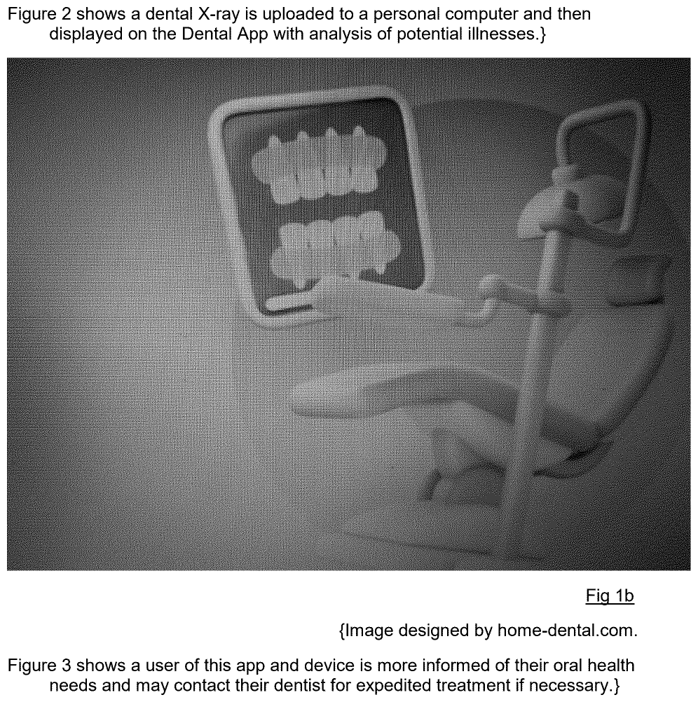

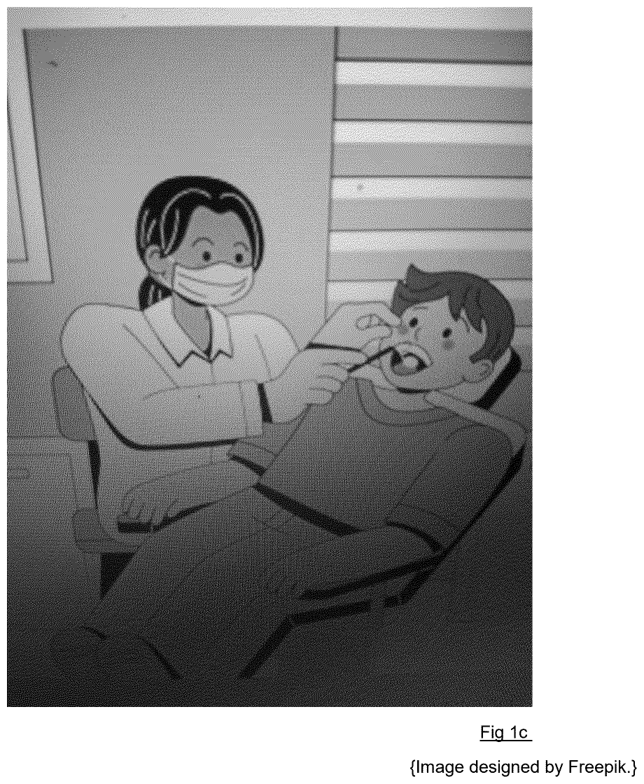

FIG. 1c shows a user of this app and device is more informed of their oral health needs and may contact their dentist for expedited treatment if necessary.
Image designed by Freepik.com.

For this “Dental App” and device innovation the financial profiteers could be:

The product manufacturers get paid on average $30,000 that includes the cost of designing, prototyping, testing, and launching the new product.

The medical insurance companies may get paid because they have more business with this preventive and diagnostic “Dental App” and device that comes from their clients having awareness of dental problems that they did not have before this product.

Stores that sell this “Dental App” and device product may split the profit between the inventor 60/40 percent with the latter getting the lessor but hope to move to 50/50.

Dental exams that include x-rays and cleaning can cost $300, and if the client does not have insurance, they might have to pay $300 out-of-pocket. This “Dental App” and device innovation may save the patient from this bill by paying for itself.

1. Designed for a personal computer. Dental X-rays from the comfort and convenience of app and device users own home.

2. For preventive and diagnostic of oral health illnesses.

3. Patients more informed of their oral health needs as they work with their dentist.
